(19) United States
(12) Patent Application Publication
(10) Pub. No.: US 2022/0079909 A1
(43) Pub. Date: Mar. 17, 2022

(54) Food Supplement For Improving Sports Performance

(71) Applicant: NUTRIBAM BV, Malle (BE)

(72) Inventor: Nikolaas VAN RIET, Halen (BE)

(21) Appl. No.: 17/421,721

(22) PCT Filed: Feb. 5, 2020
(86) PCT No.: PCT/IB2020/050914

§ 371 Date: Jul. 8, 2021

(30) Foreign Application Priority Data
Feb. 5, 2019 (BE) 2019-5071

(51) Int. Cl. A61K 31/26; A61K 31/12; A61K 36/45; A61K 36/61; A61K 36/31; A61K 31/7032; A61K 31/198; A61K 31/405; A61K 31/4172; A61K 31/205; A61K 38/06; A61K 36/67; A61K 33/06; A61K 33/00; A61K 33/42; A61K 33/24; A61K 33/30; A61K 33/22; A61K 33/26; A61K 33/32; A61K 33/04; A61K 33/34; A61K 38/47; A61P 21/06

(52) U.S. Cl. CPC A61K 31/26; A61P 21/06; A61K 36/45; A61K 36/61; A61K 36/31; A61K 31/7032; A61K 31/198; A61K 31/405; A61K 31/4172; A61K 31/205; A61K 38/063; A61K 36/67; A61K 33/06; A61K 33/00; A61K 33/42; A61K 33/24; A61K 33/30; A61K 33/22; A61K 33/26; A61K 33/32; A61K 33/04; A61K 33/34; A61K 38/47; A61K 31/12

(57) ABSTRACT

The present invention relates to the use of a composition for improving sport performances, recuperation and physiological adaptation after doing a normal physical activity, sport, exercises and/or a combination thereof, characterized in that the composition comprises sulphoraphane and/or a precursor of sulphoraphane, and curcumin as active ingredients.

### TECHNICAL DOMAIN

The invention relates to the use of a composition for the improvement of sports performances, recuperation and physiological adaptation after doing a normal physical activity, sports, exercises and/or the combination thereof.

### STATE OF THE ART

For supporting the recuperation and physiological adaptation of a user after this user has experienced a physical stress factor, the body needs amongst other things support of the endogenous antioxidant system. This system is crucial for ensuring a correct redox balance, which is necessary for an optimal physiological adaptation after exposure to a physical stress factor, such as for example a training stimulus. Free radicals (ROS & RNS, respectively Reactive Oxygen Species & Reactive Nitrogen Species) are formed under the influence of this physical stress factor and only fulfil a physiological role when they are present in very specific concentrations. However, when these specific concentrations are not met, these free radicals will fulfil a pathological role disturbing the physiological adaptation. In the past, the effect of exogenous antioxidants on the concentration of free radicals after a physical stress factor had already been examined, however always without any results. Moreover, recent studies show that the use of exogenous antioxidants can lead to a disturbed redox signalisation and a reduced physiological adaptation.

Compositions for the improvement of sports performances or recuperation are amongst other things known from US 2015 015 764 8 and US 2016 021 367 3. The described compositions and formulae would improve the human endurance during physical efforts. All these compositions have however the important disadvantage that the negative influence of exogenous antioxidants on the redox balance of the body and the additional negative effect on the recuperation and physiological adaptation of a user have not been take into account.

Compositions based on sulphoraphane and/or curcumin have been described in the literature.

Thus, Rajasri Bhattacharyya et al. describes the influence of sulphoraphane and curcumin on the expression of Nrf2. The expression of Nrf2 is amongst other things linked to a positive effect at inflammation, diabetes and neurodegenerative diseases. However, Rajasri Bhattacharyya et al doesn't describe a link between sulphoraphane and curcumin and the improvement of sports performances, recuperation and physiological adaptation. Furthermore, D1 also does not describe an influence of sulphoraphane and curcumin on the expression of NF-kB.

Shawky Noha M et al., and Kwon Jin-Sook et al., describe the influence of sulphoraphane on the expression of Nrf2 and NF-kB. However, none of the documents describes a link between sulphoraphane and curcumin and the improvement of sports performances, recuperation and physiological adaptation.

Christine A. Houghton et al., describes the influence of sulphoraphane and curcumin on the expression of Nrf2 and NF-kB and describes amongst other things the use of sulphoraphane and curcumin for use as medication for personal health management. A link between sulphoraphane and curcumin and the improvement of sports performances, recuperation and physiological adaptation is however not described.

Marco Malaguti et al., describes the protective function of a sulphoraphane treatment after an effort by rats.

Chengcao Sun et al., describes the relieving action of sulphoraphane during a muscle dystrophy treatment. Curcumin is however not mentioned.

U.S. Pat. No. 9,724,311 describes a curcumin treatment in which sports performances and endurance are improved. Sulphoraphane is however not mentioned.

EP 3 131 636 also describes compositions for improving the performances as well as the endurance for normal physical and/or sportive activities and for reducing the stress on body systems as a result of these activities. The described influence of the curcumin compositions on the performances as well as on the endurance is attributed to the modulation of the mitochondrial function; as a result, again, the redox balance of the body is not taken into account, as a result of which no optimal recuperation and physiological adaptation is possible.

There is however a need for compositions having a better influence on recovery in sportsmen.

The present invention aims to find at least a solution for some of the above-mentioned problems or disadvantages.

### SUMMARY OF THE INVENTION

In a first aspect, the present invention relates to a method for the improvement of sports performances, recuperation and physiological adaptation after doing normal physical activities, sports, exercises and/or the combination thereof in a subject, according to claim 1. Sulphoraphane as an active ingredient is an isothiocyanate, and a powerful activator of NRF2 transcription factor. Curcumin as an active ingredient is a hydrophobic polyphenol derivative and a powerful antioxidant. It is known for its diverse biological actions and pharmacological activities, such as anti-inflammatory, anti-oxidizing, anti-proliferative, antimicrobial, anti-carcinogeneous and anti-angiogenic characteristics. Curcumin inhibits the activation of the NF-κβ transcription factor. The anti-inflammatory character of curcumin at recovery is known and is linked to the inhibition of the pro-inflammatory NF-κB. Recovery of each effort is partially defined as the repair of microscopic damage to the muscle fibres. The first phase of the recovery process is always an inflammation-mediated phase. Deregulated inflammation phase by deregulated NF-KB-activations is mostly characterized by a slow recovery and can possibly lead to chronic inflammation and damage. Low recovery in sportsmen is characterized by a longer inflammation phase.

Curcumin itself thus mainly affects the inflammatory side of recovery, and also has, by means of up-regulation of SOD enzymes, a rather limited influence on the oxidative side.

Sulphoraphane, on the contrary, is described as an activator of transcription factor NRF2. NRF2 is involved in the conservation of the redox balance. This strong capacity of the NRF2 route to monitor the redox balance, is the second key for keeping the inflammation repair phase under control after sporting. Moreover, the inventors have found that activation of NRF2 antioxidant signalisation weakens the NF-KB-inflammation reaction. The combination of an NRF2 activator, with an NR-KB inhibitor seemed a powerful synergetic means at recovery of sport, namely because this combination is able of keeping a very tight control over the inflammation reaction after a great effort.

The combination of sulphoraphane or its precursor and curcumin, seems an extremely appropriate means for improving sports performances, as well as recuperation and physiological adaptation after doing normal physical activities, sport, exercises. The action of the combination means exceeds the individual action of the components.

Preferred embodiments of the method are illustrated in claims 2 to 11. The doses which have been described here, are determined for maximizing the recuperation, physiological adaptation as well as the performances of a user.

In a second aspect, the present invention relates to the use of a composition of claim 12. In particular, the composition will comprise sulphoraphane and/or a precursor of sulphoraphane, and a curcumin as active ingredients.

Preferred embodiments of the use are described in the claims 13 to 31, amongst other things relating to the ratio between sulphoraphane and glucoraphanin and the ratio between sulphoraphane and/or glucoraphanin and curcumin. These ratios are extremely important for supporting the delicate redox balance of a user, without tending towards a pathological effect. Moreover, these claims also relate to doses of the different active ingredients, for maximizing the recuperation, physiological adaptation as well as the performances of a user.

### DETAILED DESCRIPTION

The present invention relates to a composition for the improvement of sports performances, recuperation and physiological adaptation of a human being, after doing a normal physical activity, sports, exercises and/or the combination thereof.

Unless otherwise specified, all terms used in the description of the invention, including technical and scientific terms, shall have the meaning as they are generally understood by the worker in the technical field of the invention. For a better understanding of the description of the invention, the following terms are explained specifically.

The term “nutrigenomic” or “nutritional genomics” should be understood as the relation between food, the genome and the health of a subject. In particular, the term relates to the influence of one or more active ingredients to the genome expression of a subject to whom the active ingredients are administered.

The term “physiological adaptation” should be understood as processes, enabling an organism to adapt to a changing environment. These adaptations can be the result of his training, the training, or acclimatisation (for example a changing altitude, temperature, atmospheric humidity, . . . ). We speak of adaptation when a chronic physiological change occurs in the body.

Within the description of the invention, the term “extract” should be understood as a freeze-dried or spray-dried extract.

The term “curcumin” should be understood as interchangeable with the term curcuminoids. Curcuminoids comprise amongst other things the components: curcumin, methoxy curcumin, demethoxy curcumin and bisdemethoxycurcumin curcumin. Commercial products which are designated as “curcumin”, can for example comprise these components, together with the other components belonging to the class of curcuminoids. Moreover, the term “curcumin” should also be understood as comprising all derivatives and analogues of all possible components belonging to the class of curcuminoids.

The expression “% in weight” or “w/w” (percentage in weight) refers, here and throughout the description unless stated otherwise, to the relative weight of the respective component based on the total weight of the composition.

The term “physical stress factor” should be understood as doing a normal physical activity, sport, exercises and/or a combination thereof, by an organism, for example, but not limited to, a human being such as an athlete or a non-athlete.

The term “excipient” should be understood as a natural or synthetic substance formulated next to the active ingredient of the composition and is included with the aim of offering a therapeutic strengthening to the active ingredient in the final dosage form, such as the improvement of the absorption or the solubility, or as an excipient when fabricating the formulation.

The term “lubricant” should be understood as a substance which is added to a solid formulation for improving its liquidity.

The term “binding agent” should be understood as a substance keeping ingredients together, as a result of which tablets and grains can be formed.

The term “filler” or “diluent” should be understood as a substance diluting the composition or giving extra volume to the composition. A filler is generally inactive.

The term “lubricant” should be understood as a substance preventing that the finished product adheres to the surface of the tabletting apparatus.

“A”, “an” and “the” refer in the document to both the singular and the plural form unless clearly understood differently in the context. “A segment” means for example one or more than one segment.

When “approximately” or “about” are used in the document together with a measurable quantity, a parameter, a period or moment, etc., variations of +/−20% or less, preferably +/−10% or less, more preferably +/−5% or less, still more preferably +/−1% or less, and even still more preferably +/−0.1% or less than and of the cited value are meant, as far as such variations apply to the invention that is described. It will however be clearly understood that the value of the quantity at which the term “approximately” or “about” is used, is itself specified.

The terms “include”, “including”, “consist”, “consisting”, “provide with”, “contain”, “containing”, “comprise”, “comprising” are synonyms and are inclusive of open terms that indicate the presence of what follows, and that do not exclude or prevent the presence of other components, characteristics, elements, members, steps, known from or described in the state of the art.

For supporting the recuperation and physiological adaptation of a user after this user has experienced a physical stress factor, the body particularly needs support of the endogenous antioxidant system. This system is crucial for maintaining a correct redox balance, which is necessary for an optimal physiological adaptation and recuperation, after exposure to a physical stress factor such as for example a training stimulus. Under the influence of this physical stress factor, free radicals (ROS & RNS, respectively Reactive Oxygen Species & Reactive Nitrogen Species) are formed. These free radicals only fulfil a physiological role when they are produced in specific concentrations. However, in case of deviation of these specific concentrations, these free radicals will fulfil a pathological role disturbing the physiological adaptation of a user.

A composition supporting the body in maintaining a correct redox balance thus ensures a maximum physiological adaptation. In the past, the effect of exogenous antioxidants on the concentration of free radicals had already been examined, however always without any results. Moreover, recent studies show that the use of exogenous antioxidants can lead to a disturbed redox signalisation and thus also a reduced physiological adaptation. The composition according to the present invention comprises nutrigenomic components, supporting the endogenous, the body's own antioxidant system, as a result of which the required maximum physiological adaptation and recuperation is possible. Moreover, an optimized physiological adaptation and recuperation leads to a strongly improved physical fitness, which both on the long and on the short term leads to an improvement of the sport performances.

Thereto, the inventors have selected nutrigenomic components having a regulatory influence on the ‘nuclear factor-2’ (NRF2) transcription factor and the ‘nuclear factor kappa-light-chain-enhancer of activated B cells’ (NF-κβ) transcription factor. More specifically, these nutrigenomic components ensure an up-regulation of the NRF2 transcription factor and down-regulation of the NF-κβ transcription factor.

The NRF2 transcription factor is a member of the family of Cap'n'Collar transcription factors and directly influences the homeostasis of ROS and RNS free radicals, by up-regulation of the antioxidant defence system. This regulation takes place amongst other things by the activation of phase II-enzyme ‘heme oxygenase-1’ (HO-1) and the inhibition of the NF-κβ-signal path. The NF-κβ-signaling path is described as a possible cause of the pathophysiology of the muscle inflammation reaction and can amongst other things be activated by lesion-induced oxidative stress. Moreover, it regulates the promoters of several inflammatory genes.

In the present invention, a composition is described which is formulated in such way that the different components support the redox balance and that a maximum physiological adaptation and recuperation is possible. These nutrigenomic components function via the principle of hormesis, and thus ensures an adaptive reaction of a user to a physical stress factor and are, in this way, much less disruptive than exogenous antioxidants.

In a first aspect, the present invention describes a method for the improvement of sports performances, recuperation and physiological adaptation after doing a normal physical activity, sports, exercises and/or the combination thereof in a subject. In particular, a combination of sulphoraphane, and/or a derivative or precursor of sulphoraphane, and curcumin is administered to the subject.

When in the context of the present invention, it is referred to a precursor of sulphoraphane, preferably a glucoraphanin is meant.

In a second aspect, the present invention relates to a composition comprising sulphoraphane and/or a derivative or precursor of sulphoraphane, and a curcumin as an active ingredient and the use thereof. The composition is in particular appropriate for the improvement of sports performances, recuperation and physiological adaptation of a human being, after doing a normal physical activity, sports, exercises and/or the combination thereof.

The combination of curcumin and a (derivative/precursor of) sulphoraphane seemed to have a synergistic effect at the recuperation after efforts. The effect would namely be based on a positive influence on the redox balance of the body and an inhibitory effect on the inflammatory system.

In a particular embodiment, the composition and more in particular the active ingredients in the composition (sulphoraphane or derivative/precursor and curcumin) will have an effect on NRF2 transcription factor and NF-κβ transcription factor.

Sulphoraphane as an active agent is an isothiocyanate, and an activator of the NRF2 transcription factor. Studies have shown that sulphoraphane substantially improves the redox potential of the cells thanks to the activation of the NRF2 transcription factor. This improvement is due to a reduced expression of the inflammatory cytokine CD45 and the pro-inflammatory cytokines TNF-α, IL-1β and IL-6. This effect can also be mediated by the inhibition of the NF-Kκβ signaling path, which indicates that sulphoraphane has a protective role in the muscles. Moreover, sulphoraphane induces the NRF2-mediated HO-1 expression. Also, because of its strongly regulatory effect on both the NRF2 transcription factor and the NF-κβ transcription factor, sulphoraphane is an extremely important active ingredient of the composition, when a maximum physiological adaptation and recuperation is aimed for.

Sulphoraphane and/or a derivative of sulphoraphane or a sulphoraphane-precursor such as glucoraphanin can be obtained from several sources. In an embodiment, the source of sulphoraphane, its derivative or precursor is one or more plants from the Brassicaceae family. These Brassicaceae, and more in particular the different varieties of the species Brassica oleracea, belong to the preferred vegetable sources for the preparation of the composition. The family of Brassicaceae, genus Brassica, comprises amongst other things the following varieties: alboglabra (Kailan), botrytis (cauliflower), capitata (head cabbage), costata (Tronchuda borecole), gemmifera (Brussels sprouts), gongylodes (kohlrabi), italica (broccoli), medullosa (forage cale), juncea (Indian mustard), palmifolia (cavolo nero), ramosa (borecole), sabauda (savoy cabbage), sabellica (borecole) and viridis (borecole). In a possible preferred embodiment of the present invention, sulphoraphane, a sulphoraphane derivative or a precursor thereof is coming from Brassica oleracea L. var. Italica.

Sulphoraphane is also known as 1-isothiocyanato-4-methyl sulfinyl butane. Derivatives of sulphoraphane refer amongst other things, but are not limited to, sulphoxythiocarbamate analogues of sulphoraphane, 6-methyl sulfinyl hexyl isothiocyanate (6-HITC) and compounds comprising the structure of sulphoraphane with different side chains and/or different lengths of spacers between the isothiocyanato and sulfoxide groups. Examples of derivatives of sulphoraphane comprises these described in the following references: Hu et al., Eur J Med Chem, 2013, 64: 529-539; Ahn et al., Proc Natl Acad Sci USA, 2010, 107 (21): 9590-9595; and Morimistu et al., J. Biol. Chem. 2002, 277: 3456-3463 and Baird et al., Arch Toxicol, 201 1, 85 (4): 241-272.

In a preferred embodiment, the invention comprises a use of a composition, in which sulphoraphane and/or the precursor of sulphoraphane is coming from Brassica oleracea, preferably an extract and/or a powder thereof.

The precursor of sulphoraphane is glucoraphanin. In certain plants belonging to the Cruciferae, sulphoraphane occurs bound as the glucoraphanin glucosinolate. The conversion of glucosinolates in isothiocyanates in these plants is carried out via the endogenous enzyme, myrosinase, which is normally isolated in specific cell regions, but is activated when the integrity of the plant has been damaged. In humans, a partial transformation of glucosinolates in isothiocyanates takes place in the intestines by the enzymes of the residential bacterial flora. In an embodiment of the present invention, the sulphoraphane precursor glucoraphanin is used. This precursor can be obtained from any source, including, but not limited to one or more plants from the family of Brassicaceae and preferably from Brassica oleracea. In an embodiment, mustard seed or an extract of mustard seed is used as a source of myrosinase.

The use of a sulphoraphane precursor in a composition according to the present invention avoids the bitter taste of sulphoraphane in use. The predetermined total daily dose of sulphoraphane is obtained by an enzymatic conversion of glucoraphanin in sulphoraphane after consumption by a user.

In a preferred embodiment of the present invention, the used composition only comprises glucoraphanin, whether or not with addition of a source of myrosinase as described above.

In an embodiment, a combination of sulphoraphane and glucoraphanin is administered to the subject and the composition according to the present invention comprises both sulphoraphane and glucoraphanin. In another embodiment, the invention relates to a composition and its use, in which the composition comprises sulphoraphane and glucoraphanin.

A composition comprising a combination of both sulphoraphane and glucoraphanin, suppresses the bitter taste of sulphoraphane in use. This composition is in particular useful when during a production step, the composition is exposed to an increased temperature. This increased temperature reduces the enzymatic activities of myrosinase, as a result of which only a partial conversion of glucoraphanin in sulphoraphane is possible. By already providing a limited dose of sulphoraphane in the composition, the predetermined total daily dose of sulphoraphane in a user is still obtained.

In particular, the administered ratio of sulphoraphane and glucoraphanin is situated between 1:5 and 5:1, more preferably between 1:4 and 4:1, and most preferably between 1:3 and 3:1. Thereto, the composition of the present invention will comprise sulphoraphane and glucoraphanin in the ratio of 1:5 to 5:1, more preferably 1:4 to 4:1, and most preferably 1:3 to 3:1.

In a preferred embodiment, the composition of the present invention further comprises active enzymes and cofactors or one or more sources of enzymes and cofactors, which can convert glucosinolates into isothiocyanates. An example of such an enzyme is myrosinase, which can amongst other things be obtained from or which is present in plants from the family of Brassicaceae.

In particular, the total daily dose of sulphoraphane for a subject, more in particular for a human such as a sportsman, is situated between 200 μg and 60 mg, more preferably between 300 μg and 50 mg, more preferably between 400 μg and 40 mg. In another embodiment, the invention relates to a composition and its use, in which the composition is appropriate for providing a total daily dose of sulphoraphane between 200 μg and 60 mg, more preferably between 300 μg and 50 mg, more preferably between 400 μg and 40 mg.

In particular, the total daily dose of glucoraphanin for a subject, more in particular for a human such as a sportsman, is situated between 200 μg and 60 mg, more preferably between 300 μg and 50 mg, more preferably between 400 μg and 40 mg. In another embodiment, the invention relates to a composition and its use, in which the composition is appropriate for providing a total daily dose of glucoraphanin between 200 μg and 60 mg, more preferably between 300 μg and 50 mg, more preferably between 400 μg and 40 mg.

In particular, a combination of sulphoraphane and glucoraphanin will be administered in which the total maximum daily dose of sulphoraphane and glucoraphanin for a subject, more in particular a human such as a sportsman, is approximately 120 mg, preferably approximately 100 mg, and most preferably approximately 80 mg. In another embodiment, the invention relates to a composition and its use, in which the composition is appropriate for providing a combination of sulphoraphane and glucoraphanin with a total maximum daily dose of approximately 120 mg, preferably approximately 100 mg, and most preferably approximately 80 mg.

These doses seemed to support the delicate redox balance of a user, without tending towards a pathological effect.

Curcumin as an active ingredient is a hydrophobic polyphenol derivative, belonging to the class of the diarylheptanoids. Curcumin is a powerful antioxidant and can be obtained from the spices curcuma. It is known for its diverse biological actions and pharmacological activities, such as anti-inflammatory, anti-oxidizing, anti-proliferative, antimicrobial, anti-carcinogeneous and anti-angiogenic characteristics. Curcumin inhibits the activation of the NF-κβ transcription factor regulating a number of genes coding for pro-inflammatory cytokines (TNF-alpha, IL-1, IL-6), chemokines (IL-8, MIPI-alpha), inducible enzymes (iNOS, COX-2) and other molecules. Thus, curcumin regulates the inhibition of the NF-βκ transcription factor, the production of phospholipase, lipo-oxygenase, cyclo-oxygenase 2, leukotrienes, thromboxane, prostaglandins, nitric oxide (NO), collagenase, elastase, hyaluronidase, monocyte chemoattractant protein-1 (MCP-1), interferon-inducible protein, TNF-alpha and IL-12. Moreover, curcumin for example also increases the activity of amongst other things superoxidedismutase (SOD) and glutathione peroxidase (GSH). Both enzymes play a very important role in our endogenous antioxidant system, which again shows the extremely large importance of curcumin as an active ingredient in a composition aiming for a maximum physiological adaptation and recuperation.

In an embodiment, the used curcumin in the composition or the method will come from Curcuma longa, preferably an extract and/or powder thereof.

Optionally and additionally, the composition can also comprise diarylheptanoids with comparable or the same biological characteristics and effects as curcumin.

In a particular embodiment, sulphoraphane and/or a derivative or precursor of sulphoraphane and curcumin will be administered to a subject in one single composition.

In a particular embodiment, the total daily dose of curcumin will be situated between 30 mg and 300 mg, more preferably between 40 mg and 250 mg, and most preferably between 60 mg and 200 mg. In another embodiment, the composition of the present invention will be appropriate for providing a total daily dose of curcumin between 30 mg and 300 mg, more preferably between 40 mg and 250 mg, and most preferably between 60 mg and 200 mg.

These doses seemed to support the delicate redox balance of a user, without tending towards a pathological effect.

In a particular embodiment, sulphoraphane and/or a derivative or precursor thereof and curcumin will be administered in a ratio situated between 1:1 and 1:50, more preferably 1:2 to 1:40, and more preferably 1:5 to 1:30. The composition of the present invention will thereto comprise a ratio of sulphoraphane and/or a derivative or precursor thereof and curcumin situated between 1:1 and 1:50, more preferably 1:2 to 1:40, and more preferably 1:5 and 1:30.

In a particular embodiment, sulphoraphane and curcumin will be administered in a ratio situated between 1:1 and 1:50, more preferably 1:2 to 1:40, and more preferably 1:5 to 1:30. The composition of the present invention will thereto comprise a ratio of sulphoraphane and curcumin situated between 1:1 and 1:50, more preferably 1:2 to 1:40, and more preferably 1:5 to 1:30.

In a further embodiment, glucoraphanin and curcumin will be administered in a ratio situated between 1:1 and 1:50, more preferably 1:2 to 1:40, and more preferably 1:5 to 1:30. The composition of the present invention will thereto comprise a ratio of glucoraphanin and curcumin situated between 1:1 and 1:50, more preferably 1:2 to 1:40, and more preferably 1:5 and 1:30.

In a further embodiment, the composition will comprise Vaccinum, sect. Cyanococcus (blue berry) or a derivative thereof as an active ingredient. In particular, Vaccinium sect. Cyanococcus will be selected from the group of Vaccinium myrtillus, Vaccinium uliginosum, Vaccinium caespitosum, Vaccinium deliciosum, Vaccinium membranaceum, Vaccinium ovalifolium, Vaccinium angustifolium, Vaccinium boreale, Vaccinium caesariense, Vaccinium cyanococcus, Vaccinium corymbosum, Vaccinium darrowii, Vaccinium elliottii, Vaccinium fuscatum, Vaccinium hirsutum, Vaccinium myrtilloides, Vaccinium operium, Vaccinium myrtilloides, Vaccinium pallidum, Vaccinium simulatum, Vaccinium tenellum, Vaccinium virgatum and Vaccinium formosum, preferably Vaccinium myrtillus and Vaccinium Cyanococcus. In a particularly preferred embodiment, the composition comprises Vaccinium myrtillus.

Preferably, the daily dose of Vaccinium, sect. Cyanococcus will be situated between 1 g and 5 g, more preferably between 2 g and 4 g, and most preferably between 2.5 g and 3.5 g. In a preferred embodiment, the composition of the present invention will be formulated in such way that it is appropriate for providing a daily amount of Vaccinium, sect. Cyanococcus as described herein.

These daily doses seemed ideal for improving sports performances, since such Vaccinium, sect. Cyanococcus species are very rich in exogenous antioxidants. The daily dose of Vaccinium, sect. Cyanococcus, as described in the present invention, is configured for supporting the delicate redox balances of a user. This additional support for the exogenous antioxidants is particularly important for offering the desired physiological adaptation and recuperation.

In a particular embodiment, the composition will comprise Vaccinum, sect. Cyanococcus and/or Syzygium aromaticum.

In a further or another embodiment, the composition comprises a Syzygium aromaticum or a derivative thereof as an active ingredient. In an embodiment, the daily dose of Syzygium aromaticum will be situated between 0.3 g and 1 g, more preferably 0.4 g to 0.9 g, and more preferably between 0.5 g and 0.8 g. Preferably, the composition of the present invention will be formulated in such way that it is appropriate for providing a daily amount of Syzygium aromaticum as described herein.

This dose seemed ideal for improving sport performances, since Syzygium aromaticum have antiseptic and anaesthetic characteristics, playing an important role in the recuperation of a user, after the influence of a physical stress factor. Active ingredients, playing an important role here, are for example: eugenol, beta-caryophylline, vanillin, crategolic acid, methyl salicylate, tannines, flavonoids (such as eugenine, kaempferol, rhamnetin and eugenitin), triterpenoids (such as oleanolic acid, stigmasterol and campesterol) and different sesquiterpenes. Preferably, Syzygium aromaticum is added to the composition in the form of a freeze-dried extract or in the form of a powder.

In an embodiment, a composition of the present invention comprises at least:
Glucoraphanin
Curcumin
And optionally, a source of myrosinase such as for example mustard seed or an extract of mustard seed.

Glucoraphanin is preferably present in the composition in a concentration allowing administration of a daily dose situated between 200 μg and 60 mg, while curcumin is present in a concentration allowing administration of a daily dose situated between 30 mg and 300 mg.

Next to the (optional) presence of mustard seed or an extract thereof, the composition can further be supplemented with Vaccinum, sect. Cyanococcus in a daily dose situated between 1 g and 5 g, and/or Syzygium aromaticum in a daily dose situated between 0.3 g and 1 g.

In a further or another embodiment of the present invention, the composition comprises at least:
a daily dose of sulphoraphane between 200 μg and 60 mg, and/or
a daily dose of glucoraphanin situated between 200 μg and 60 mg, and
a daily dose of curcumin between 30 mg and 300 mg, and
a daily dose of Vaccinium, sect. Cyanococcus situated between 1 g and 5 g, and
a daily dose of Syzygium aromaticum between 0.3 g and 1 g.

In a further embodiment, the invention comprises a use of a composition as described in one of the embodiments, in which the composition comprises Piper nigrum or a derivative thereof as an active ingredient. Preferably, the daily dose of Piper nigrum comprises 5 mg to 50 mg, more preferably 10 mg to 40 mg, and most preferably 20 mg to 30 mg.

Piper nigrum comprises approximately 4% to 10% of piperine. This piperine increases the bioavailability of active ingredients in the composition such as curcumin, which causes a synergetic effect between the different components.

In a further embodiment, the composition comprises a concentrate or extract selected from the group of Withania somnifera, Phyllanthus emblica, Piper nigrum or a combination thereof.

Compositions according to the present invention can additionally and/or optionally further comprise one or more free amino acids or peptides, such as lysine, methionine, histidine, leucine, isoleucine, alanine, phenylalanine asparagine, arginine, beta-alanine, aspartic acid, tryptophan, proline, threonine, cysteine, selenocysteine, serine, taurine, tyrosine, valine, glycine, glutamine, glutamic acid, ornithine, carnosine, L-carnitine, glutathione.

Compositions according to the present invention additionally and/or optionally further comprise one or more minerals selected from the group of calcium, sodium, potassium, phosphorus, chromium, vanadium, magnesium, zinc, manganese, selenium, copper, molybdenum, boron, vanadium, iron (and/or derivatives) or a combination thereof.

According to an embodiment of the invention, the composition comprises a dosage form for oral administration. Examples of dosage forms for oral administration comprise, but are not limited to, a tablet, a capsule, powder which can be dispersed in a drink, a liquid such as a solution, suspension or emulsion, a soft gel/chewing rod or any other appropriate dosage form known in the state of the art. Preferably, the composition is formulated in powder form, as a tablet, a capsule or a granule. Moreover, the dosage forms for oral administration can be formulated for immediate release, prolonged release or delayed release.

Compositions according to the present invention can additionally and/or optionally further comprise one or more excipients. These excipients are selected from the following group: microcrystalline cellulose, crystalline cellulose, lactose, corn starch, sucrose, glucose; binding agents such as tragacanth, Arabic gum, corn starch, gelatine, polyvinyl alcohol, polyvinyl ether, ethyl cellulose, methyl cellulose, shellac, hydroxypropyl cellulose, hydroxypropyl starch, polyvinyl pyrrolidon; swelling agents such as corn starch, pregelatines starch, alginic acid, dextrin; lubricants such as magnesium stearate; fluidity-improving agents such as fine silica dioxide; lubricants such as glyceryl fatty acid ester, magnesium stearate, talc, polyethylene glycol, silica, hydrogenated vegetable oil; sweeting agents such as sucrose, lactose, aspartame, acesulfam-K, sucralose, monatine, stevia, saccharine and similar; tastes which have to be used for different food such as peppermint, vanilla taste, cherry, raspberry ketone and similar; conservatives such as paraoxybenzoates, chlorobutanol, benzyl alcohol, sorbic acid; an antioxidant, such as sulphite, ascorbic acid, vitamin E, butyl hydroxy toluene, sodium sulphite; and/or coating agents such as shellac, sucrose, gelatine and hydroxypropyl cellulose.

Compositions of the present invention can additionally and/or optionally further comprise phytochemical molecules, coming from at least one of the following sources: blueberry, cranberry, raspberry, cherry, mulberry, pomegranate, maqui berry, purple corn, strawberry, grapes, gooseberry, black berries, grapes must, cacao beans, coffee beans, pine-tree bark, cardamom, cinnamon bark, ginseng, astragalus, rhodiola, garcinia, ginger, ginkgo, citrus fruits, grape skins, grape pits, hawthorn, artichoke, apple, olive, orange, lemon, pepper, soy, mango, tea leaves, tomato, cabbage, black rice, bitter lemon, stevia, lo han, goji (wolf cherry), sea buckthorn, kudzu, clove, hemp, cassia, magnolia, nutmeg, jujube, honeysuckle, poria, bellflower, lotus, basil, sesame, angelica, cimicifuga, Epimedium, Schisandra, salvia, liquorice, privet, ophiopogonis, aloe, dodder, fenugreek, gotu kola, guarana, purslane and Tribulus.

Compositions of the present invention can additionally and/or optionally further comprise one or more additional natural extracts, selected from one or more plants from the following genus: Origanum Thymus, Lavandula, Salvia, Melissa, Cuminum, Petroselinum, Calendula, Tagetes, Boswellia, Sambucus, Copaifera, Allium, Symphytum, Punica, Euterpe, Sophora, Rheum, Fagopyrum, Camellia, Coptis, Hydrastis, Mahonia, Phellodendron, Berberis, Xanthorhiza, Lonicera, Vaccinium, Cinnamomum, Vitis, Terminalia, Pinus, Albizia, Melia, Salvadora, Paullinia, Commiphora, Juglans, Scutellaria and Magnolia

In particular, the additional natural extract which is used in the compositions described here, can be extracted from plants from the following species: Origanum vulgare, Origanum onites, Origanum majorana, Origanum heracleoticum, Thymus vulgaris L, Thymus citriodorus, Thymus pulegioides, Thymus x herba-barona, Thymus serpyllum, Lavandula angustifolia/officinalis, Lavandula stoechas, Lavandula dentate, Lavandula x intermedia, Lavandula multifida, Salvia officinalis, Salvia divinorum, Salvia apiana, Melissa officinalis, Cuminum cyminum, Petroselinum crispum, Calendula arvensis, Calendula maderensis, Calendula officinalis, Tagetes erecta, Tagetes minuta, Tagetes patula, Boswellia sacra, Boswellia frereana, Boswellia serrata, Boswellia papyrifera, Sambucus nigra, Sambucus melanocarpa, Sambucus racemosa, Copaifera langsdorfii, Curcuma longa, Allium sativu, Symphytum officinale, Punica granatum, Euterpe oleracea, Sophora flavescens, Rheum rhabarbarum, Rheum rhaponticum, Fagopyrum esculentum, Camel lia sinensis, Coptis teeta, Hydrastis canadensis, Mahonia aquifolium, Phellodendron amurense, Berberis vulgaris, Xanthorhiza simplicissima, Lonicera ceprifoliu, Vaccinium macrocarpon, Cinnamomum zeylanicum Nees, Cinnamomum verum, Vitis Vinifera, Terminalia Bellerica, Pinus Pinaster, Albizia Lebbek, Melia Azadirachta, Salvadora persica, Paullinia cupana, Piper betle, Commiphora myrrha, Juglans regia, Scutellaria baicalensis and Lombardia officinalis.

Additional natural extracts that can be useful together with predefined active ingredients, can also be chosen from one or more of the following natural extracts (general name first included, not cursive, followed by the official cursive name (names)): achyranthes, Achyranthes aspera, aloe, Aloë spp., including A. barbadensis, A. feroxand A. vera, anise, Pimpinella anisum, aristolochia, Aristolochia bracteolate, arnica, Arnica spp., including A. fulgens, banyan, Ficus bengalensis, bakula, Mimusops elengi, basil, Ocimum basilicumand O. minimum, betle, Piper betle, camphor, Cinnamomum camphora, catechu, Acacia catechu,g greater celandine, Chelidonium spp., camomile, Matricaria chamomilla, chebula, Terminalia chebula, Chinese calotte, Scutellaria baicalensis, cinnamon, Cinnamomum lourerii and C. zeylandicum, citrus, Citrus spp., such as C. aurantifolia, C. aurantium, C. limonumand, C. sinensis, dill, Anethum spp., including A. graveolens and A. sowa, echinacea (sun hat), Echinacea pallida, eucalyptus, Eucalyptus globulus, fennel, Foeniculum vulgare, gardenia, Gardenia jasminoides, grape, Vitis vinifera, hop, Humulus lupulus, houttuynia, Houttuynia cordata, Indian mulberry, Morinda citrifolia, juniper berry, Juniperus communis, lemongrass, Cymbopogon spp., including C. citratus and C. flexuosus, liquorice, Glycyrrhiza spp., including G. labra and G. uralensis, long pepper (pipli), Piper longum, madhuca, Madhuca longifolia, magnolia, Magnolia officinalis, marigold, Calendula officinalis, mastic, Pistacia lentiscus, melilotus, Melilotus officinalis, water milfoil, Achillea millefolium, myrrh, Commiphora spp., including C. abyssinica and C. molmol, neem (margosa), Azadirachta indica, neroli (bitter orange blossom), Citrus aurantium, nutmeg, Myristica fragrans, oak gall, Quercus infectoria, parsley, Petroselinum sativum, peelu, Salvadora persica, pepper mint, Mentha piperita, pine-tree, Pinus spp., including P. palustris and P. sylvestris, pomegranate, Punica granatum, prickly acacia (babul), Acacia nilotica, rhatany, Krameria spp., including K argentea and K triandra, rosemary, Rosmarinus officinalis, saffron, Crocus sativus, sage, Salvia spp., including S. lavendulae folia, S. officinalis and S. triloba, sandalwood, Santalum spp., including S. album and S. spicatum, spearmint Mentha spicata, spilanthes (akarkara), Spilanthes calvi, steranijs, Illicium verum, tea (including green tea and oolong tea), Camellia sinensis, thyme, Thymus spp., including T. serpyllum and T vulgaris, tomar (prickly ash), Zanthoxylum armatum, tulsi (holy basil), Ocimum sanctum, usnea, Usnea barbata, vajradanti, Potentillafulgens, walnut, Juglans regia, wintergreen, Gaultheria procumbence and mixtures thereof.

As described here, the additional natural extracts can be derived from or be based on compounds or extracts isolated from plants.

Additionally en/or optionally, compositions and formulations of the present invention can further comprise one or more vitamins, selected from the group of: vitamin A, vitamin C, vitamin B1, vitamin B2, vitamin B3, vitamin B5, vitamin B6, vitamin B12, vitamin D, vitamin E, vitamin K (and/or derivatives), folic acid, biotin.

Additionally and/or optionally, compositions of the present invention can further comprise one or more additives, such as fruit juice, dextrin, cyclic oligosaccharide, cyclodextrins (alpha, beta, gamma), saccharides (monosaccharides such as fructose, glucose and/or polysaccharides), buffers, acidulant, flavourings, Hiki cha powder or other flavour modifiers, an emulsifier, collagen, milk powder, polysaccharide thickener, agar or other texture modifiers, at least one vitamin, egg shell calcium, calcium pantothenate, other minerals, queen jelly, propolis honey, food fibres, Agaricus subrufescens, chitin, chitosan, flavonoids, carotenoids, lutein, herb medicine, chondroitin and/or amino acids.

Compositions of the present invention can also comprise creatine and derivatives thereof, capsate, carnitine, nordihydrocapsate, pterostilbene and other polyphenols and agmatine (or agmatine sulphate).

Additionally and/or optionally, compositions of the present invention can further comprise one or more citrus bioflavonoids, (such as quercetin, rutine, isoquercetin, synephrine and octopamine); CoQ10, thiamine, citrulline malate, nicotinamide adenoside dinucleotide (NAD), nicotinamide riboside (NR), citrulline, lutein, lycopene, capsaicin, arginine alpha ketoglutarate (Arginine AKG), L-arginine pyroglutamate, arginine ketoisocaproate, citric acid, ornithine alpha ketoglutarate (Ornithine AKG), omega-3 fatty acids (DHA and EPA), L-norvaline, nitrate, taurine, arginine ethyl ester, carnosine, vanadyl sulphate, L-alpha glycerophosphoryl choline (Alpha GPC), Pinus pinaster (Pycnogenol®), rutacaerpine, Epimedium spp., garlic (allicin, alliin and similar), linseed, linseed ligas (alpha-linolenic acid (ALA), gamma-linolenic acid (GLA)), Schisandria (such as Schisandrin, Schisandrol A and B and Gamma schisandrin), green tea catechins (such as catechin, ECGC, ECG and EGC), black tea, dong quai (ligustilide), Andrographis (Andrographolides), grape extract (such as resveratrol) anthocyanines (such as cyanidin 3-glucosi de (C3G), cyanidin 3-rutinoside, delphinidin 3-glucoside and malvidin 3-glucoside), Danshen, Beta vulgaris carrot, celery (3-N-butylftalide), Berberine, mother herb, jasmine, lemon balm, vinpocetine, Lotus, Malrove, Lemongrass, Yerba mate, Peony, Mustard, Motherwort, Cramp-bark, Grapeseed, Proanthocyanidines (PAC's, such as Procyanidin A1, Procyanidin A2, Procyanidin B1, Procyanidin B2, and similar), spinach (with nitrates), Kale (with nitrates), Beet (with nitrates), theobromine, theophylline, phenylethylamine (PEA) and similar), hawthorn, Hawthorn-flavonoids (hyperoside, vitexin, isovitexine and similar), Catauba-extract, apple polyphenols and combinations thereof.

Additionally and/or optionally, compositions of the present invention can further comprise one or more NO-inducing agents, such as glyceryl trinitrate (also known as nitro-glycerine), isosorbide mononitrate, isosorbide dinitrate, clonitrate, ettriol trinitrate (ETTN), erythrityl tetranitrate, pentaerythritol tetranitrate (PETN), pentrinitrol, D-mannitol hexanitrate, trolnitrate phosphate, sodium nitroprusside, PDES-inhibitors (slidenafil, tadalafil, vardenafil), papaverine, bamethan, bencyclane, beraprost, betahistine, brovincamine, bufeniode, buflomedil, butalamine, cetiedil, chromonar, ciclonicate, cinepazide, cinnarizine, clobenfurol, cloricromen, cyclandelate, dilazep, droprenilamine, eburnamonine, efloxate, eledoisine, etafenon, fasudil, fendiline, fenoxedil, flunarizine, hexobendine, ibudilast, ifenprodil, iloprost, inositol niacinate, itramin tosylate, kallidin, kallikrein, khellin, lidoflazine, lomerizine, moxisylyte, nafronyl nicotinylalcohol, nimodipine, nylidrin, pentifylline, pimefylline, piribedil, trapidil, trimetazidine, vincamine, vinpocetine, viquidil, visnadine, xanthinol niacinate, bendazol, floredil, medibazine, tinofedrine, amotriphene, benfurodil hemisuccinate, hepronicate, hepronicate, nicofuranose, suloctidil, or salts and/or prodrugs thereof.

Additionally and/or optionally, compositions and formulations of the present invention can further comprise one or more carrier agents, such as corn starch, acacia, gelatine, malt, tragacanth, microcrystalline cellulose, kaolin, dicalcium phosphate, calcium carbonate, sodium chloride or alginic acid; disintegrating agents such as microcrystalline cellulose or alginic acid; binding agents such as acacia, methyl cellulose, ethyl cellulose, sodium carboxymethylcellulose, polyvinylpyrrolidone or hydroxypropyl methylcellulose; and lubricants such as magnesium stearates, stearic acid, silicon liquid, talc, oils, waxes or colloidal silica.

Additionally and/or optionally, compositions and formulations of the present invention can further comprise one or more mixtures of acids, (such as citric acid, tartaric acid, malic acid and fumaric acid or a combination thereof) and carbonates such as sodium, potassium bicarbonate or carbonate; water-soluble binding agents (starches, natural gums, cellulose gum, microcrystalline cellulose, methylcellulose, cellulose-ethers, ethyl cellulose, sodium carboxymethylcellulose, gelatine, dextrose, lactose, sucrose, sorbitol, mannitol, polyethylene glycol, polyvinylpyrrolidone, pectins, alginates, polyacrylamides, polyvinyl oxoazolidon, polyvinyl alcohols and mixtures thereof) and/or lubricants (sodium benzoate, polyethylene glycol, L-leucine, adipic acid and combinations thereof).

Additionally and/or optionally, compositions and formulations of the present invention can further comprise one or more other ingredients, such as for example flavourings, filling agents, surfactants, colouring agents and sweeteners.

Additionally and/or optionally, compositions and formulations of the present invention can further comprise one or more other water or other watery formulations, such as suspending agents such as for example alginates, pectin, kelgin, carrageenan, acacia, methyl cellulose, polyvinyl alcohol or polyvinyl pyrrolidon.

Additionally and/or optionally, compositions and formulations of the present invention can further comprise one or more, watery or non-watery carriers, diluents, filling agents, binding agents, humidifiers, disintegrating agents, solubility-retarding agents, absorption catalysts, humidifying agents, absorbing agents and lubricant, solvents or carriers, including water-ethanol, polyols (propene glycol, polyethylene glycol or glycerol), appropriate mixtures thereof, vegetable oils (such as olive oil) and organic esters such as ethyl oleate.

Furthermore, the compositions and formulations of the present invention can comprise stabilizers, solubility-enhancers, suspending agents, emulsifiers, tranquillizers, buffers, conservatives, isotonic agents or antibacterial and antifungal agents.

In a preferred embodiment of the present invention, the composition will be a composition formulated as a powder, preferably a water-soluble powder.

In particular, the composition will comprise between 2 g and 10 g of curcumin per 100 g of the composition, more preferably between 3 and 8 g of curcumin per 100 g and between 0.25 g and 5 g of sulphoraphane and/or glucoraphanin per 100 g of the composition, more preferably between 0.4 and 3 g of sulphoraphane and/or glucoraphanin per 100 g of the composition.

In a preferred embodiment, the composition will comprise only glucoraphanin and no sulphoraphane. In this preferred embodiment, the composition will comprise between 1.00 and 0.5 g of mustard seed extract per 100 g of composition, in which the extract is a source of myrosinase.

Further ingredients can amongst other things be selected from Piper nigerum, lecithin, alpha cyclodextrin, vitamins, minerals, spore elements and plant extracts and derivatives as described above.

### EXAMPLES

The invention will now be further described by means of the following example, without being limited thereto.

### Example 1

In the last year, a sportsman active in the world of mountain biking achieved on average a 50th place in World Cup competitions. After taking a composition according to an embodiment of the present invention, comprising sulphoraphane and curcumin as an active ingredient, the sport performances improved. As a result, he always ended up in the top 20. No other changes in the eating pattern were observed, the other aspects of his training also remained unchanged. This sudden and remarkable progress indicated a strongly changed fitness which was achieved thanks to the nutrigenomic support. As a result, the training volume was also strongly increased, which indicates a strong physiological adaptation and an improved and faster recuperation. The daily dose of sulphoraphane was 30 mg, the daily dose of curcumin was 75 mg.

Similar results were obtained when sulphoraphane was replaced by the precursor glucoraphanin, whether or not in the presence of a source of myrosinase.

### Example 2

A simple composition of the present invention relates to a composition with one or more sources of sulphoraphane or precursor glucoraphanin and curcuma.

Examples of further possible compositions of the present invention are amongst other things.

| Composition | Composition | Composition | Composition | Composition |
|---|---|---|---|---|
| 1 | 2 | 3 | 4 | 5 |
| Sulphoraphane | Sulphoraphane | Sulphoraphane | Sulphoraphane | / |
| (1- | (1- | (1- | (1- | |
| isothiocyanato- | isothiocyanato- | isothiocyanato- | isothiocyanato- | |
| 4-methyl | 4-methyl | 4-methyl | 4-methyl | |
| sulfinyl | sulfinyl | sulfinyl | sulfinyl | |
| butane) 10 mg | butane) 60 mg | butane) 30 mg | butane) 600 μg | |
| Glucoraphanin | Glucoraphanin | Turmeric | Glucoraphanin | Glucoraphanin |
| (4-methyl | (4-methyl | Longa extract | (4-methyl | (4-methyl |
| sulfinyl butyl) | sulfinyl butyl) | (curcumin) 70 | sulfinyl butyl) | sulfinyl butyl) |
| extract 15 mg | extract 45 mg | mg | extract 3 mg | extract 23 mg |
| Curcuma | Curcuma | Piper Nigrum | Curcuma | Curcuma |
| Longa extract | Longa extract | (piperine) 25 mg | Longa extract | Longa extract |
| (curcumin) 75 mg | (curcumin) | | (curcumin) 30 mg | (curcumin) |
| | 195 mg | | | 200 mg |
| Piper Nigrum | Piper Nigrum | Syzygium | Piper Nigrum | Piper Nigrum |
| (piperine) 10 | (piperine) 50 | aromaticum | (piperine) 50 | (piperine) 50 |
| mg | mg | 250 mg | mg | mg |
| Syzygium | Syzygium | Cyanococcus | Syzygium | Syzygium |
| aromaticum | aromaticum | (blue berry) | aromaticum 1 g | aromaticum 1 g |
| 190 mg | 850 mg | 575 mg | | |
| Cyanococcus | Cyanococcus | Brassica | Cyanococcus | Cyanococcus |
| (blue berry) | (blue berry) | oleracea | (blue berry) | (blue berry) |
| 350 mg | 1.5 g | extract - | 5 g | 5 g |
| | | 10 mg | | |
| Brassica | Brassica | citric acid | Paullinia | Paullinia |
| juncea extract - | juncea extract - | 10 mg | cupana - | cupana - |
| 35 mg | 230 mg | | 183.2 mg | 183.2 mg |
| Withania | Humulus | Vaccinium | Vaccinium | Vaccinium |
| somnifera 4 | lupulus 50 mg | macrocarpon | macrocarpon | macrocarpon |
| mg | | 10 mg | 70 mg | 70 mg |
| Phyllanthus | Citric acid | Paullinia | Citric acid | Citric acid |
| emblica (Amla) | 10 mg | cupana 10 mg | 200 μg | 200 μg |
| 1 mg | | | | |
| Glycine 1 mg | Carnosine 1 | Histidine 1 mg | Histidine 2 mg | Histidine 2 mg |
| | mg | | | |
| Ornithine 1 mg | Ornithine 2 mg | Leucine 1 mg | Leucine 1 mg | Leucine 1 mg |
| Selenium 1 mg | Manganese 1 mg | Carnosine 1 mg | Manganese 1 mg | Manganese 1 mg |
| Magnesium 1 mg | Magnesium 2 mg | Calcium 5 mg | Calcium 5 mg | Calcium 5 mg |
| Manganese 1 mg | Manganese 3 mg | Manganese 1 mg | Molybdenum 1 mg | Molybdenum 1 mg |
| Zinc 5 mg | Potassium 1 mg | Phosphor 1 mg | Phosphor 3 mg | Phosphor 3 mg |
| Example of | Example of | Example of | Example of | Example of |
| composition 1 | composition 2 | composition 3 | composition 4 | composition 5 |
| is administered | is formulated | is administered | is formulated | is formulated |
| orally in the | as a powder, | orally in the | as a powder, | as a powder, |
| form of a | which can be | form of a | which can be | which can be |
| granule, with a | dispersed in a | tablet, with a | dispersed in a | dispersed in a |
| total weight of | drink. Every | total weight of | drink. Every | drink. Every |
| 700 mg. This | day, 3 g of the | 1 g. This tablet | day, 6.35 g of | day, |
| granule should | described | should be | the described | minimum 7 g |
| be | powder should | administered | powder should | of the |
| administered | be | twice a day. | be | described |
| three times a | administered. | | administered. | powder |
| day. | | | | should be |
| | | | | administered. |

Another embodiment comprises a powdery composition, appropriate for dissolution or dispersion in a liquid, comprising at least the following ingredients:

Per 100 grams of composition:

3 grams of Curcuma longa (95% of curcumin)

35 grams Brassica oleracea (3.5 g Sulphoraphane Glucosinolates/Glucoraphanin)

0.025 grams of mustard seed extract

0.03 grams of Piper nigerum (95% of piperine)

The composition is further supplemented with excipients such as amino acids, vitamins, spore elements, minerals and other vegetable derivatives.

Preferably, 5 to 10 grams of the composition is dosed in an amount of liquid such as water.

### Example 3

The efficiency of a composition of the present invention, comprising sulphoraphane and curcumin as an active ingredient, was analysed. This analysis was realized, for monitoring the performances and recuperation of subjects, after doing a daily physical activity. The monitoring was done by means of a training diary which had to be completed three times a day, as well as by measuring the ‘heart rate variability’ (HRV), and the ‘rest hear rate’ (RHR) of the subjects. The composition was administered orally every day in the form of a powder or was dispersed in a drink. The daily dose of this powder comprises 50 mg of sulphoraphane, 50 mg of curcumin and 900 mg of Gatorade powder.

The HRV was analysed under controlled laboratory circumstances, before and after doing the prescribed daily physical activity. Analogously, the RHR was analysed under controlled laboratory circumstances, before and after doing the prescribed daily physical activity. The training diary was made up of a questionnaire, which needed to be completed by means of a scale of five (scale of Linkert). Questions were asked about possible muscle pain of the subjects, but also for example their energy level during the different moments of the day.

Fifty male subjects were selected to take part in the experiment. Their physical characteristics were as follows (average and SD): age 25.1 SD:2.7 years; weight 82.4 SD:3.3 kg; length 180 SD:3 cm. Each subject was asked to take a daily dose for five successive days. Each dose was dissolved in hot water. During this period, the subjects also had to follow a similar protein diet. Two weeks after completing this protocol, the same protocol was repeated with a similar dose of placebo, thereto Gatorade powder was used.

During this 5-day period, the subjects who had received the composition, indicated having much less muscle pain and less fatigue during the day. Moreover, the HRV indices show that a subject will overtrain less, when this subject takes a daily dose of the prescribed composition.

After three days, a significant reduction in the RHR of the subjects is also observed, compared to the placebo group. These results show the positive influence of the prescribed composition, both on the recuperation and on the sport performances.

### Example 4

The synergetic efficiency of a composition comprising both sulphoraphane and curcumin, was analysed. This analysis was realized, for monitoring the performances and recuperation of subjects, after doing a daily physical activity. The monitoring was done by means of a training diary which had to be completed three times a day, as well as by measuring the ‘heart rate variability’ (HRV), and the ‘rest hear rate’ (RHR) of these subjects.

The HRV was analysed under controlled laboratory circumstances, before and after doing the prescribed daily physical activity. Analogously, the RHR was analysed under controlled laboratory circumstances, before and after doing the prescribed daily physical activity. The training diary was made up of a questionnaire, which needed to be completed by means of a scale of five (scale of Linkert). Questions were asked about possible muscle pain of the subjects, but also for example their energy level during the different moments of the day.

37 male subjects were selected to take part in the experiment. Their physical characteristics were as follows (average and SD): age 27.6 SD:1.6 years; weight 79.3 SD:2.1 kg; length 184 SD:3 cm. The subjects were divided into 4 groups: a group receiving only curcumin (Group C, n=10) (daily dose of 50 mg), a group receiving only sulphoraphane (Group S, n=10) (daily dose 25 mg), a group receiving both curcumin and sulphoraphane (Group C+S, n=10) (the same concentrations as the controls), and finally, a negative control was included in the experiment, it comprises a group receiving a similar dose of placebo, thereto Gatorade powder was used (Group nC, n=7). Each subject was asked to take a prescribed daily dose for five successive days. Each dose was dissolved in hot water. During this period, the subjects also had to follow a similar protein diet. The whole experiment was divided into 4 time periods, here, the different groups were tested successively. Between the different time periods, a two-week period was provided in which no experiment was carried out.

The different compositions were administered orally every day in the form of a powder or dispersed in a drink. The daily dose of this powder for Group C comprised 25 mg of curcumin and 950 mg of Gatorade powder. The daily dose of this powder for Group S comprised 50 mg of sulphoraphane and 950 mg of Gatorade powder. The daily dose of this powder for Group C+S comprised 25 mg of sulphoraphane, 50 mg of curcumin and 950 mg of Gatorade powder. The daily dose of this powder for Group nC comprised 1000 mg of Gatorade powder.

During this 5-day period, the C+S group indicated suffering less from muscle pain and fatigue during the day, a significant difference was observed compared to all other groups (Group S, Group C and Group nC). For both Group C and Group S, less muscle pain and fatigue were also observed, compared to the negative control (Group nC). The HRV and RHR indices moreover show that subjects from groups C +S, C and S will probably overtrain less compared to subjects from the negative control (Group nC). Moreover, a significant difference in the HRV and RHR indices was observed between Group C+S and the groups C and S separately. These results show both the efficiency of sulphoraphane and curcumin separately, and the synergetic effect of both active components in combination.

The tests were repeated with doses as described above and also with other ratios.

1. A method for improving sport performances, recuperation and physiological adaptation after doing a normal physical activity, sport, exercises and/or a combination thereof in a subject, comprising administering a combination of sulphoraphane and/or a derivative or precursor of sulphoraphane and curcumin to the subject.

2. The method of claim 1, characterized in that sulphoraphane is administered to a subject, with a total daily dose of sulphoraphane situated between 200 μg and 60 mg.

3. The method of claim 1, characterized in that the precursor of sulphoraphane is glucoraphanin, with a total daily dose of glucoraphanin situated between 200 μg and 60 mg.

4. The method of claim 1, characterized in that the precursor of sulphoraphane is glucoraphanin and wherein a combination of sulphoraphane and glucoraphanin is administered.

5. The method of claim 1, characterized in that a daily dose of curcumin is situated between 30 mg and 300 mg.

6. The method of claim 1, characterized in that sulphoraphane and/or the derivative or precursor of sulphoraphane and curcumin is administered to the subject in one single composition.

7. The method of claim 1, characterized in that a ratio of sulphoraphane and/or the derivative or precursor of sulphoraphane and curcumin is situated between 1:1 and 1:50.

8. The method of claim 4, characterized in that the composition further comprises sulphoraphane and a ratio of sulphoraphane and glucoraphanin is situated between 1:5 and 5:1.

9. The method of claim 4, characterized in that the composition comprises sulphoraphane and a total maximum daily dose of sulphoraphane and glucoraphanin of approximately 120 mg.

10. The method of claim 6, characterized in that the composition comprises sulphoraphane and a ratio of sulphoraphane and curcumin is situated between 1:1 and 1:50.

11. The method of claim 6, characterized in that the precursor of sulphoraphane is glucoraphanin and wherein the composition comprises glucoraphanin and a ratio of glucoraphanin and curcumin is situated between 1:1 and 1:50.

12. The method of claim 1, in which the composition further comprises Vaccinium, sect. Cyanococcus and/or Syzygium aromaticum.

13. Use of a composition for improving sport performances, recuperation and physiological adaptation after doing a normal physical activity, sport, exercises and/or a combination thereof, characterized in that the composition comprises sulphoraphane and/or a derivative or precursor of sulphoraphane and curcumin as active ingredients.

14. The use of a composition of claim 13, characterized in that sulphoraphane and/or the precursor of sulphoraphane is coming from Brassica oleracea.

15. The use of a composition of claim 13, characterized in that the precursor of sulphoraphane is glucoraphanin and wherein the composition comprises sulphoraphane and glucoraphanin.

16. The use of a composition of claim 13, characterized in that the curcumin is coming from Curcuma longa.

17. The use of a composition of claim 13, characterized in that a ratio of sulphoraphane and/or a derivative or precursor thereof and curcumin is situated between 1:1 and 1:50.

18. The use of a composition of claim 15, characterized in that a ratio of sulphoraphane and glucoraphanin is situated between 1:5 and 5:1.

19. The use of a composition of claim 13, characterized in that a ratio of sulphoraphane and glucoraphanin is situated between 1:1 and 1:50.

20. The use of a composition of claim 15, characterized in that a ratio of glucoraphanin and curcumin is situated between 1:1 and 1:50.

21. The use of a composition of claim 13, characterized in that the composition is appropriate for offering a total daily dose of sulphoraphane between 200 μg and 60 mg.

22. The use of a composition of claim 15, characterized in that the composition is appropriate for offering a total daily dose of glucoraphanin situated between 200 μg and 60 mg.

23. The use of a composition of claim 15, characterized in that the composition comprises is appropriate for offering a total maximum daily dose of sulphoraphane and glucoraphanin of approximately 120 mg.

24. The use of a composition of claim 13, characterized in that the composition is appropriate for offering a total daily dose of curcumin between 200 μg and 300 mg.

25. The use of a composition of claim 13, characterized in that the composition comprises a Vaccinium, sect. Cyanococcus or a derivative thereof as an active ingredient, selected from the group of Vaccinium myrtillus, Vaccinium uliginosum, Vaccinium caespitosum, Vaccinium deliciosum, Vaccinium membranaceum, Vaccinium ovalifolium, Vaccinium angustifolium, Vaccinium boreale, Vaccinium caesariense, Vaccinium cyanococcus, Vaccinium corymbosum, Vaccinium darrowii, Vaccinium elliottii, Vaccinium fuscatum, Vaccinium hirsutum, Vaccinium myrtilloides, Vaccinium operium, Vaccinium pallidum, Vaccinium simulatum, Vaccinium tenellum, Vaccinium virgatum and Vaccinium formosum.

26. The use of a composition of claim 25, characterized in that a daily dose Vaccinium, sect. Cyanococcus, comprises 1 g to 5 g.

27. The use of a composition of claim 13, characterized in that the composition comprises a Syzygium aromaticum or a derivative thereof as an active ingredient, and the a daily dose of Syzygium aromaticum comprises 0.3 g to 1 g.

28. The use of a composition of claim 13, characterized in that the composition comprises:
a daily dose of sulphoraphane between 200 μg and 60 mg, and/or
a daily dose of glucoraphanin situated between 200 μg and 60 mg, and
a daily dose of curcumin between 30 mg and 300 mg, and optionally
a daily dose of Vaccinium, sect. Cyanococcus situated between 1 g and 5 g, and
a daily dose of Syzygium aromaticum between 0.3 g and 1 g;
wherein the precursor of sulphoraphane is glucoraphanin.

29. The use of a composition of claim 13, characterized in that the composition comprises Piper nigrum or a derivative thereof as an active ingredient.

30. The use of a composition of claim 13, characterized in that the composition comprises one or more amino acids and/or peptides, selected from the group of lysine, methionine, histidine, leucine, isoleucine, alanine, phenylalanine asparagine, arginine, beta-alanine, aspartic acid, tryptophan, proline, threonine, cysteine, selenocysteine, serine, taurine, tyrosine, valine, glycine, glutamine, glutamic acid, ornithine, carnosine, L-carnitine, glutathione or a combination thereof.

31. The use of a composition of claim 13, characterized in that the composition comprises one or more minerals selected from the group of calcium, sodium, potassium, phosphorus, chromium, vanadium, magnesium, zinc, manganese, selenium, copper, molybdenum, boron, vanadium, iron (and/or derivatives) or a combination thereof.

32. The use of a composition of claim 13, characterized in that the composition comprises is formulated in a powder form, as a tablet, a capsule or a granule.

33. The use of a composition of claim 13, characterized in that the composition has an effect on NRF2 transcription factor and NF-κβ transcription factor.

34. A composition comprising sulphoraphane and/or glucoraphanin, curcumin and optionally a source of myrosinase.

35. The composition of claim 34, characterized in that the composition is formulated as a powder.

36. The composition of claim 34, characterized in that the composition comprises between 2 g and 10 g of curcumin per 100 g of composition and between 0.25 g and 5 g of sulphoraphane and/or glucoraphanin per 100 g of composition.

37. The composition of claim 34, characterized in that the composition only comprises glucoraphanin.

38. The composition of claim 34, characterized in that the composition comprises between 0.01 g and 0.5 g of mustard seed extract per 100 g of composition.
